(19) United States
(12) Patent Application Publication
(10) Pub. No.: US 2021/0177259 A1
(43) Pub. Date: Jun. 17, 2021

(54) SYSTEM AND METHOD FOR CACHING AND PROCESSING SENSOR DATA LOCALLY

(71) Applicant: Aetna Inc., Hartford, CT (US)

(72) Inventors: Ajay Behuria, Hartford, CT (US); Alan Bachman, Hartford, CT (US); Manu Marulachary, Hartford, CT (US); Anthony J. Cevoli, Hartford, CT (US)

(21) Appl. No.: 16/717,306

(22) Filed: Dec. 17, 2019

(51) Int. Cl. A61B 5/00; G16H 40/67; A61B 5/11; H04L 29/08; A61B 5/0205

(52) U.S. Cl. CPC A61B 5/0022; G16H 40/67; A61B 5/0205; H04L 67/10; A61B 5/1118

(57) ABSTRACT

Methods and systems for analyzing care data using an edge computing device are provided. The edge computing device is connected to a cloud service. The edge computing device receives a machine learning algorithm from the cloud service. The edge computing device receives first care data from a first sensor and second care data from a second sensor. The edge computing device analyzes the first care data to obtain a first care data score and analyzes the second care data to obtain a second care data score. Next, the edge computing devices scores, using the machine learning algorithm, the first care data score and the second care data score to obtain a combined care score. The edge computing device determines whether the combined care score is greater than a threshold. The edge computing device triggers an emergency procedure when it is determined that the combined care score is greater than the threshold.

### BACKGROUND OF THE INVENTION

Many sensor devices are not powerful enough to perform advanced computations on their own. For example, a fitness tracker or other sensor may have limited processing power to update and display step counts and other information. However, the sensor may not have the processing power to perform complex machine learning tasks. Additionally, data from various sensors is typically siloed and not available for processing by other sensors. For example, a fitness tracker may not have access to data from an air quality sensor. Therefore, sensor data is often processed in isolation from other sensor data. This limits the usefulness of the collected data.

Performing sensor computations using cloud computing may allow more advanced processing to be accomplished. However, cloud computation is typically high latency and provides delayed time response. In certain regulated applications, such as healthcare, cloud computing has certain restrictions regarding dependability, privacy concerns and regulations.

At the same time, society is increasingly relying on deployment of myriads of sensors for monitoring and providing details about a state of the environment and the health of individuals. The sensors may be networked and deployed in vehicles, homes, offices, and other locations. Sensors can also be deployed on individuals and even embedded in individuals.

### BRIEF SUMMARY OF THE INVENTION

One embodiment provides a method for analyzing care data using an edge computing device. The method includes registering the edge computing device. The edge computing device is connected to a cloud service. The edge computing device receives a machine learning algorithm from the cloud service. The edge computing device receives first care data from a first sensor and second care data from a second sensor. The edge computing device analyzes the first care data to obtain a first care data score and analyzes the second care data to obtain a second care data score. Next, the edge computing devices scores, using the machine learning algorithm, the first care data score and the second care data score to obtain a combined care score. The edge computing device determines whether the combined care score is greater than a threshold. The edge computing device triggers an emergency procedure when it is determined that the combined care score is greater than the threshold.

In another embodiment, an edge computing device is provided. The edge computing device includes a processor; and a non-transitory computer readable medium storing instructions, that when executed by the processor cause the edge computing device to perform steps. The steps include registering the edge computing device. The edge computing device is connected to a cloud service. The edge computing device receives a machine learning algorithm from the cloud service. The edge computing device receives first care data from a first sensor and second care data from a second sensor. The edge computing device analyzes the first care data to obtain a first care data score and analyzes the second care data to obtain a second care data score. Next, the edge computing devices scores, using the machine learning algorithm, the first care data score and the second care data score to obtain a combined care score. The edge computing device determines whether the combined care score is greater than a threshold. The edge computing device triggers an emergency procedure when it is determined that the combined care score is greater than the threshold.

In yet another embodiment a non-transitory computer readable medium storing instructions, that when executed by a processor cause the processor to perform steps is provided. The steps include registering the edge computing device. The edge computing device is connected to a cloud service. The edge computing device receives a machine learning algorithm from the cloud service. The edge computing device receives first care data from a first sensor and second care data from a second sensor. The edge computing device analyzes the first care data to obtain a first care data score and analyzes the second care data to obtain a second care data score. Next, the edge computing devices scores, using the machine learning algorithm, the first care data score and the second care data score to obtain a combined care score. The edge computing device determines whether the combined care score is greater than a threshold. The edge computing device triggers an emergency procedure when it is determined that the combined care score is greater than the threshold.

### BRIEF DESCRIPTION OF THE SEVERAL VIEWS OF THE DRAWING(S)

FIG. 1 illustrates a system diagram of an edge computing environment according to an embodiment;

FIG. 2 is a flow diagram of a method for configuring an edge computing device according to an embodiment;

FIG. 3 is a flow diagram of a method for deploying machine learning algorithms on an edge computing device according to an embodiment;

FIG. 4 is a flow diagram of a method for analyzing sensor data using an edge computing device according to an embodiment;

FIG. 5 is a flow diagram illustrating a process for scoring data from various sensors according to an embodiment;

FIG. 6 illustrates a system diagram of an edge computing environment for detecting and responding to epilepsy seizures according to an embodiment; and

FIG. 7 illustrates a computing device according to an embodiment.

### DETAILED DESCRIPTION OF THE INVENTION

Sensors are increasingly used for monitoring and providing details about a state of the environment and the health of individuals. Sensors can be deployed in vehicles, homes, offices, and other locations. Sensors can also be deployed on individuals and even embedded in individuals. Embodiments described herein provide time sensitive and responsive healthcare management by leveraging edge computing, data and network resources physically located closer to the source of the information such as healthcare sensors and actuators.

The use of edge computing reduces privacy concerns and increases dependability. Because an edge computing device may be owned and/or controlled by an individual, the individual is better able to manage their own data which reduces privacy concerns. Further, reducing or eliminating the need to send sensor data to a cloud computing environment reduces network latency and increases dependability of the system.

Embodiments provide more ubiquitous and cost-effective healthcare data and information to individuals including patients and healthcare plan members. Additional, embodiments provide critical care monitoring to individuals suffering from chronic conditions.

The described systems may perform data aggregation, data filtering, artificial intelligence and real-time data analytics at the edge using an edge computing device. This allows for more effective engagement with individuals, to better understand the context, and to predict and respond to health emergencies.

The described systems and methods enable near real-time or real-time data analysis with improved bandwidth efficiency and faster application response times. Further, the described systems are able to cope with intermittent network connectivity.

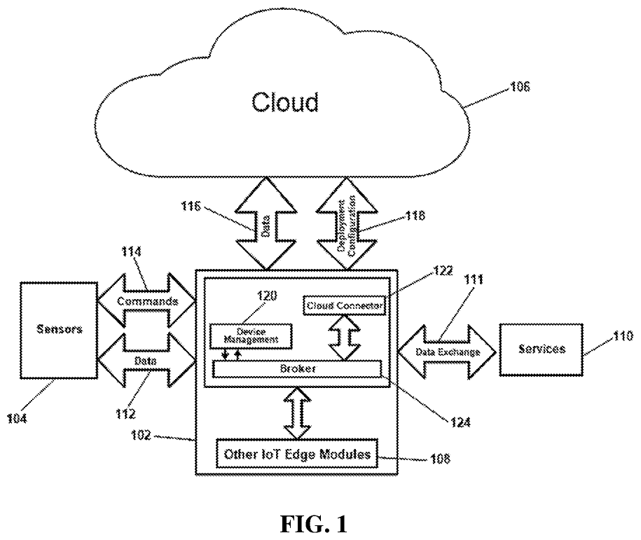

Turning to the figures, FIG. 1 illustrates a system diagram of an edge computing environment according to an embodiment. In this embodiments, edge computing device 102 connects to sensors 104 and cloud computing resources 106. The cloud computing resources 106 include cloud services. Sensors 104 can include one or more sensors for collecting environmental data or data from an individual. Example sensors for collecting data from an individual include a smart ring, smart glasses, a smart shirt, a smart watch, Bluetooth tracker, smart shoes, smart socks, smart pants, a smart belt, an Simultaneous GPS (SGPS)/General Packet Radio Service (GRPS) baby control, a smart bracelet, and a smart finger. A smart finger may be a wearable device, or a device implanted in the user. For example, the wearable or implanted device may continuously measure how a person's fingernail bends and moves, which is an indicator of grip strength. Grip strength is a useful metric in a broad set of health issues. It has been associated with the effectiveness of medication in individuals with Parkinson's disease, the degree of cognitive function in schizophrenics, the state of an individual's cardiovascular health, and all-cause mortality in geriatrics.

Environmental sensors may include air quality sensors, smoke detectors, and temperature sensors. The sensors can gather various pieces of data including heart rate, body temperature, movement, geographic location, elevation, step count, number of stairs climbed, blood oxygen level, and more.

Edge computing device 102 can send and receive data from sensors 104 through data channel 112. Edge computing device 102 can also send and receive commands from sensors 104 through command channel 114. The edge computing device 102 communicates with sensors using any appropriate network connection, such as Bluetooth, Wi-Fi, cellular and others.

Edge computing device 102 interfaces with various internet of things (IOT) modules through connection 108. Example IoT modules at the edge include modules to deidentify patient data prior to sending it to the cloud. Patient data may be deidentified for privacy and security reasons. Additional examples include modules for data aggregation from multiple sensors, modules for data filtration, and modules for synchronous/asynchronous messaging between the modules at the edge computing device.

Additionally, the edge computing device 102 may interface with various emergency and non-emergency services 110 though a data exchange 111. These service 110 may include a traffic management center, a pharmacy, a electronic health record, an electronic medical record, emergency services such as police, fire and medical personal, paramedics, emergency care providers and health insurers. Other services 110 may include informational services such as weather services. The edge computing device 102 can both send and receive data from the services using various cellular, Wi-Fi and other networks.

Edge computing device 102 also interfaces with cloud computing resources 106. The cloud computing resources may include third party resources or may be hosted by the entity providing the edge computing device 102. Additionally, cloud computing resources 106 may also be a computing device owned or controlled by the individual the is using the edge computing device.

The edge computing device 102 can send and receive data from the cloud computing resources 106 through data channel 116. Edge computing device 102 can also send and receive commands and deployment configuration information through channel 118. Device management at the edge requires providing configuration information, updates and patches to the edge device. The deployment configuration information is referring to these types of device management related instructions and payloads. For example, an edge computing device and sensors may be provided to a user. Deployment configuration information may be used to provide the initial configuration for the edge computing device and sensors.

The edge computing device 102 can be worn by an individual, carried by an individual, or installed at a location. For example, the edge computing device could be a mobile computing device, such as a mobile phone or tablet computer worn by an individual. In some embodiments, the edge computing device 102 may be a wearable computer, such as a high power smart watch. In other embodiments, the edge computing device may be a computer, router or media device at an individual's home or work. Any computing device with appropriate processing power and network connectivity could be the edge computing device.

The edge computing device 102 includes a number of software and hardware modules for connectivity and processing. For example edge computing device 102 may include a device management module 120. The device management module 120 interfaces with the various sensors 104 that are connected to or may connect to the edge computing device 102. The device management module 120 may monitor and track sensor state, and provide a rule engine for processing and scoring data from the sensors. This process is described below.

The edge computing device 102 also includes a cloud connector 122. The cloud connector 122 connects to the various public and private cloud computing resources 106. A broker 124 interfaces the device management module 120, the cloud connector 122 and the other IoT modules through connection 108 together. In this embodiment, data and commands to and from the sensors 104 is sent through the device management module 120, data and commands to and from the cloud computing resources 106 is sent through the cloud connector 122.

In some embodiments the edge computing device is not always or always expected to be connected to the cloud computing resources through the network. In such cases, the edge computing device, such as a mobile computing device will intermediately connect to the cloud computing resources and exchange data when connected. For example, mobile computing device may connect to the network using a cellular connection.

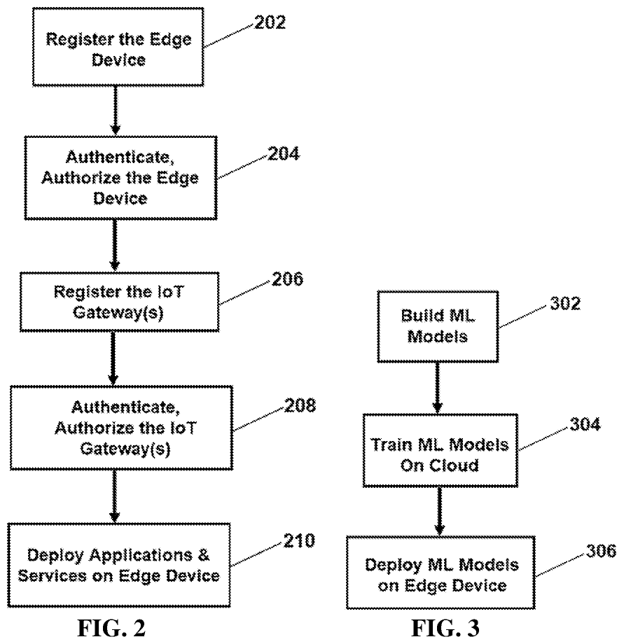

FIG. 2 is a flow diagram of a method for configuring an edge computing device according to an embodiment. At step 202, the edge device is registered. Step 202 may include registering the edge computing device with the cloud computing resources. At step 204, the edge computing device is authenticated, and the edge computing device is authorized. For example, organization identification, device type, device identification, and an authentication token may be provided to configure the edge device and connect to the cloud computing resources. At step 206, any gateways, such as IoT gateways are registered. The gateways are managed devices to that may connect to an IoT platform, such as cloud computing resources. In one embodiment, the gateway is the edge computing device or a component in the edge computing device, such as the cloud connector 202 in FIG. 1. In this embodiment, the gateway is registered at the time the edge computing device is registered in step 202.

At step 208, the gateway device is authenticated, and the gateway device is authorized. For example, organization identification, device type, device identification, and an authentication token may be provided to configure the edge device and connect to the cloud computing resources. In one embodiment, the gateway is the edge computing device or a component in the edge computing device, such as the cloud connector 202 in FIG. 1. In this embodiment, the gateway is registered at the time the edge computing device is registered in step 204. At step 210, applications and services are deployed on the edge computing device. The applications and services may be implemented on the edge device using native code or may be implemented in container packages, such as a docker container or KubeEdge. The applications and services may be used to obtain the sensor data and analyze the data as described in more detail below. The applications and services deployed on the edge computing device may be responsible for all or a portion of its functionality. Example functionality includes connecting to sensors, analyzing sensor data, connecting to cloud services and contacting emergency service providers.

FIG. 3 is a flow diagram of a method for deploying machine learning algorithms on an edge computing device according to an embodiment. At step 302, the machine learning models and algorithm are built in the cloud. In some embodiments, the machine learning models are specific to a particular chronic condition. For example, in order to predict cardiac arrest, the machine learning model will monitor sensor data for blood pressure, accelerometer, ECG, activity, fall detection, weather conditions, GPS location, acoustics and other relevant parameters. As described below, if a combined score for the parameters is greater than a threshold then care providers may be notified. The machine learning models for every chronic condition may be different. For example, diabetes, epilepsy, parkinson's Disease, and COPD will each have different machine learning models.

The cloud trains the machine learning model at step 304. The model may be trained using data from various edge computing devices and other sources of data. At step 306, the machine learning model and algorithm is deployed to the edge computing device. The machine learning model and algorithm may be implemented on the edge device using native code or may be implemented in containers, such as a docker container or KubeEdge. Thus, at step 306, the edge computing device receives a machine learning algorithm from the cloud.

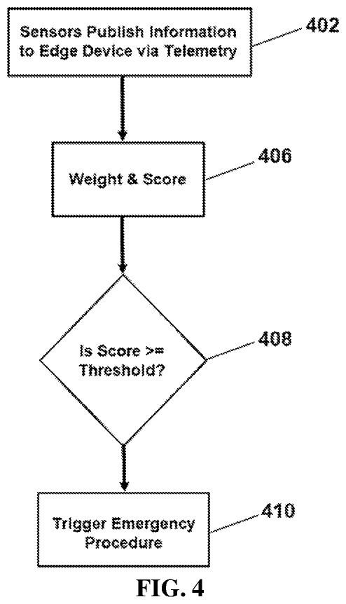

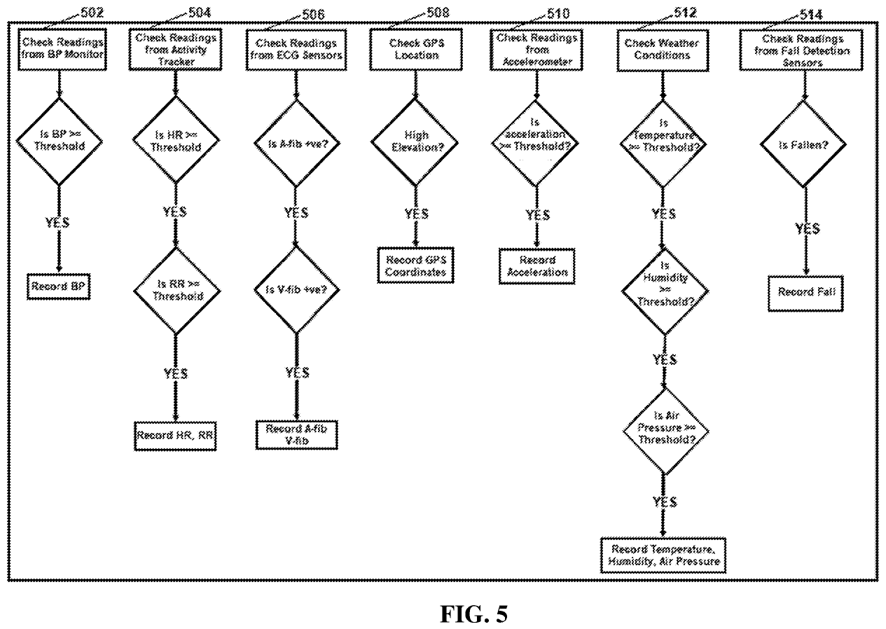

FIG. 4 is a flow diagram of a method for analyzing sensor data using an edge computing device according to an embodiment. At step 402, the sensors publish information and data to the edge computing device. For example, at step 402, the edge computing device may receive first care data from a first sensor and second care data from a second sensor. At step 406, the sensor data is weighted and scored. For example, at step 406, the edge computing device may analyze the first care data to obtain a first care data score. Further, the edge computing device may analyze the second care data to obtain a second care data score. In some embodiments, the edge computing device scores, using the machine learning algorithm, the first care data score and the second care data score to obtain a combined care score. FIG. 5, below, further describes weighting and scoring sensor data in some embodiments.

At step 408, the edge computing device determines whether the score is equal to or exceeds a threshold. In one embodiment, the edge computing device determines whether the combined care score is greater than a threshold. At step 410, an emergency procedure is triggered if the score is equal to or exceeds a threshold. For example, the edge computing device may trigger an emergency procedure when it is determined that the combined care score is greater than the threshold. In some embodiments, an emergency procedure includes automatically contacting an emergency service provider. In other embodiments, a care provider or other person is first contacted to determine whether an emergency service provider should be contacted.

FIG. 5 is a flow diagram illustrating a process for scoring data from various sensors according to an embodiment. For example, after the edge computing device receives data from a sensor, it can score the data immediately, or at a later time. At process 502, readings from a blood pressure monitor are analyzed. If the blood pressure exceeds a threshold, the blood pressure reading is recorded by the edge computing device. At process 504, readings from a activity tracker are analyzed. In one embodiment, the activity tracker includes a heart rate monitor. If the heart rate exceeds a threshold, the heart rate reading is recorded by the edge computing device. Alternatively, if the heart rate exceeds a threshold, the edge computing device determines if the resting heart rate variability exceeds a threshold. If the heart rate variability exceeds a threshold, then the heart rate and heart rate variability are recorded.

At process 506, readings from an electrocardiogram (ECG) sensor are analyzed. If the ECG reading indicates atrial fibrillation, the atrial fibrillation incident is recorded. Alternatively, if the ECG reading indicates ventricular fibrillation, the ventricular fibrillation is recorded. In one embodiment if both atrial fibrillation and ventricular fibrillation are indicated, both readings are recorded.

At process 508, readings from a global positioning system (GPS) are read. If the altitude reading from the GPS exceeds a threshold, the altitude data and/or the GPS coordinates are recorded by the edge computing device. At process 510, readings from an accelerometer, such as a fitness tracker or smartwatch are read. If the acceleration reading from the accelerometer exceeds a threshold, the acceleration is recorded by the edge computing device.

At process 512, weather conditions are checked. Weather conditions can be checked through an online service or through a weather monitoring device. In either case, they may be referred to as a weather sensor. If the temperature exceeds a threshold, the temperature is recorded. If the humidity exceeds a threshold, the humidity is recorded. If the air pressure exceeds a threshold, the air pressure is recorded. In some embodiments all three parameters must exceed the thresholds before they are recorded. At process 514, readings from a fall detection sensor is read. If the fall detection sensor indicates a fall, the fall is recorded by the edge computing device. Readings from various exemplary sensors are shown in FIG. 5. However, any combination of sensors can be used with embodiments of this disclosure. The sensors used will depend on the application.

After being recorded, the sensor data is then scored. For example, in one embodiment data is scored on a four-point system, from 0-3. A higher score indicates a deteriorating condition. For example, a blood pressure monitor may assign a 0 to systolic blood pressure between 101-199 mm Hg and a 2 to pressure greater than 200 mm Hg. A heart rate monitor may assign 0 to heart rate readings between 51-100 beats/min, 1 to heart rate readings between 101-110 beats/min, 2 to heart rate readings between 111-129 beats/min and 3 to heart rate readings between 130 beats/min. These ranges are just given as examples.

As discussed above, with respect to FIG. 4, the edge computing device may generate a combined score using the individual sensor data scores and the machine learning algorithm. The machine learning model and algorithm may be built in the cloud using the cloud computing resources. In some embodiments, the edge computing device transmits to the cloud service on the cloud computing resources, deidentified first care data, such as the sensor data. For example, the cloud service may modify the machine learning algorithm using deidentified first care data and deidentified second care data. The edge computing device could then receive the modified machine learning algorithm from the cloud.

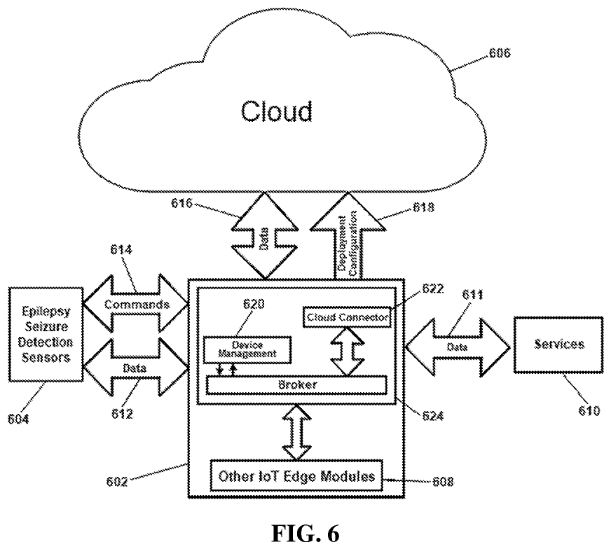

FIG. 6 illustrates a system diagram of an edge computing environment for detecting and responding to epilepsy seizures according to an embodiment. This embodiment uses the systems and methods described above to detect and respond to epileptic seizures. The edge computing device 602 is registered with cloud computing resources 606 and is connected to at least one cloud service running on the cloud computing resources 606. The edge computing device 602 then receives a machine learning algorithm from the cloud computing resources 606. Information and data are exchanged between the cloud computing resources 606 and the edge computing device 602 using the data channel 616 and the cloud connector 622. Further, the edge computing device 602 can also send and receive commands and deployment configuration information through channel 618.

In this embodiment, the edge computing device 602 connects to various sensors 604 for detecting an epileptic seizure. Example sensors include sensors 604 for recording the wearer's movements, such as a accelerometer, gyroscope and/or compass. The sensors 604 can be embedded in an activity tracker, smart clothing, smart watch or other device. The device management module 620 facilitates communication with the sensors 604. The edge computing device 602 sends and receives data with the sensors 604 over data channel 612 and sends and receives commands over command channel 614. In some embodiments the data channel 612 and the command channel 614 are the same channel.

The edge computing device 602 receives data from the sensors 604 including, for example, first care data from a first sensor and second care data from a second sensor. As described above, the edge computing device then analyzes the sensor data to obtain a data score. For example, the edge computing device 602 analyzes the first care data to obtain a first care data score and the second care data to obtain a second care data score. The edge computing device 602 the scores the sensor data using the machine learning algorithm to generate a combined care score. For example, edge computing device 602 the scores the first care data score and the second care data score to obtain a combined care score.

Next, the edge computing device 602 determines whether the combined care score is greater than a threshold. In this embodiment, if the combined care score exceeds the threshold, an epileptic seizure may be in progress or recently occurred. If the combined score is greater than a threshold, the edge computing device 602 an emergency procedure. For example. The edge computing device may contact a service 610, such as an emergency service, through data channel 611. A broker 624 interfaces the device management module 620, the cloud connector 622 and the other IoT modules through connection 608 together. This facilitates communication between the various entities.

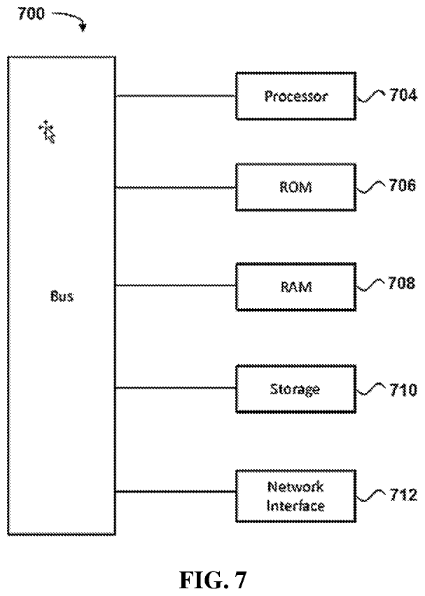

FIG. 7 illustrates a computing device according to an embodiment. The computing device 700 can be used to implement the sensors, edge computing device, cloud computing resources and other devices described above. The computing device 700 includes a processor 704, such as a central processing unit (CPU), executes computer executable instructions comprising embodiments of the system for performing the functions and methods described above. In embodiments, the computer executable instructions are locally stored and accessed from a non-transitory computer readable medium, such as storage 710, which may be a hard drive or flash drive. Read Only Memory (ROM) 706 includes computer executable instructions for initializing the processor 704, while the random-access memory (RAM) 708 is the main memory for loading and processing instructions executed by the processor 704. The network interface 712 may connect to a wired network, wireless network or cellular network and to a local area network or wide area network, such as the internet.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for analyzing care data using an edge computing device, the method comprising:
registering the edge computing device;
connecting the edge computing device to a cloud service;
receiving, by the edge computing device, a machine learning algorithm from the cloud;
receiving, by the edge computing device, first care data from a first sensor and second care data from a second sensor;
analyzing, by the edge computing device, the first care data to obtain a first care data score;
analyzing, by the edge computing device, the second care data to obtain a second care data score;
scoring, by the edge computing device using the machine learning algorithm, the first care data score and the second care data score to obtain a combined care score;
determining, by the edge computing device, whether the combined care score is greater than a threshold; and
triggering, by the edge computing device, an emergency procedure when it is determined that the combined care score is greater than the threshold.

2. The method of claim 1, further comprising:
transmitting, by the edge computing device to the cloud service, deidentified first care data and deidentified second care data, wherein the cloud service modifies the machine learning algorithm using the deidentified first care data and deidentified second care data.

3. The method of claim 2, further comprising:
receiving, by the edge computing device, the modified machine learning algorithm from the cloud.

4. The method of claim 1, wherein the triggering an emergency procedure comprises automatically contacting an emergency service provider.

5. The method of claim 1 further comprising transmitting, by the edge computing device to the cloud service, deployment configuration data.

6. The method of claim 1 wherein the edge computing device is a mobile computing device.

7. The method of claim 1 wherein the edge computing device connects to the first sensor using a cellular data connection.

8. The method of claim 1 wherein the first sensor is one of a blood pressure monitor, an activity tracker, an electrocardiogram sensor, a global positioning system device, an accelerometer, a weather monitor, and a fall detection sensor.

9. The method of claim 1 wherein the edge computing device connects to the first sensor using a service in a container package.

10. The method of claim 9 wherein the container package is a docker container.

11. An edge computing device comprising:
a processor; and
a non-transitory computer readable medium storing instructions, that when executed by the processor cause the edge computing device to perform steps comprising:
registering the edge computing device;
connecting the edge computing device to a cloud service;
receiving, by the edge computing device, a machine learning algorithm from the cloud;
receiving, by the edge computing device, first care data from a first sensor and second care data from a second sensor;
analyzing, by the edge computing device, the first care data to obtain a first care data score;
analyzing, by the edge computing device, the second care data to obtain a second care data score;
scoring, by the edge computing device using the machine learning algorithm, the first care data score and the second care data score to obtain a combined care score;
determining, by the edge computing device, whether the combined care score is greater than a threshold; and
triggering, by the edge computing device, an emergency procedure when it is determined that the combined care score is greater than the threshold.

12. The method of claim 11, further comprising:
transmitting, by the edge computing device to the cloud service, deidentified first care data and deidentified second care data, wherein the cloud service modifies the machine learning algorithm using the deidentified first care data and deidentified second care data.

13. The method of claim 12, further comprising:
receiving, by the edge computing device, the modified machine learning algorithm from the cloud.

14. The method of claim 11, wherein the triggering an emergency procedure comprises automatically contacting an emergency service provider.

15. The method of claim 11 further comprising transmitting, by the edge computing device to the cloud service, deployment configuration data.

16. The method of claim 11 wherein the edge computing device is a mobile computing device.

17. The method of claim 11 wherein the edge computing device connects to the first sensor using a cellular data connection.

18. The method of claim 11 wherein the first sensor is one of a blood pressure monitor, an activity tracker, an electrocardiogram sensor, a global positioning system device, an accelerometer, a weather monitor, and a fall detection sensor.

19. The method of claim 11 wherein the edge computing device connects to the first sensor using a service in a container package.

20. A non-transitory computer readable medium storing instructions, that when executed by a processor cause the processor to perform steps comprising:
registering the edge computing device;
connecting the edge computing device to a cloud service;
receiving, by the edge computing device, a machine learning algorithm from the cloud;
receiving, by the edge computing device, first care data from a first sensor and second care data from a second sensor;
analyzing, by the edge computing device, the first care data to obtain a first care data score;
analyzing, by the edge computing device, the second care data to obtain a second care data score;
scoring, by the edge computing device using the machine learning algorithm, the first care data score and the second care data score to obtain a combined care score;
determining, by the edge computing device, whether the combined care score is greater than a threshold; and
triggering, by the edge computing device, an emergency procedure when it is determined that the combined care score is greater than the threshold.
